SEQUENCE LISTING (3 sequences)

SEQ ID NO: 1 - protein, 20 aa, Artificial Sequence
  Synthetic Construct
KIPKACCVPTELSAISMLYL

SEQ ID NO: 2 - protein, 21 aa, Artificial Sequence
  Synthetic Construct
CKXPKXSSXPTEXSAISMLYL

SEQ ID NO: 3 - protein, 20 aa, Artificial Sequence
  Synthetic Construct
KXPKXXXXPTEXSAISMLYL

(19) United States
(12) Patent Application Publication
(10) Pub. No.: US 2016/0022875 A1
(43) Pub. Date: Jan. 28, 2016

(54) BONE SUBSTITUTES GRAFTED BY MIMETIC PEPTIDES OF HUMAN BMP 2 PROTEIN

(71) Applicant: TEKNIMED, VIC-EN-BIGORRE (FR)

(72) Inventors: Marie-Christine DURRIEU, BORDEAUX (FR); Omar ZOUANI, BORDEAUX (FR)

(73) Assignee: TEKNIMED, VIC-EN-BIGORRE (FR)

(21) Appl. No.: 14/775,181

(22) PCT Filed: Mar. 14, 2014
(86) PCT No.: PCT/FR2014/050601

§ 371 Date: Sep. 11, 2015

(30) Foreign Application Priority Data
Mar. 14, 2013 (FR) 1352302

(51) Int. Cl. A61L 27/54; A61K 6/02; A61L 27/22

(52) U.S. Cl. CPC A61L 27/54; A61L 27/227; A61K 6/02; A61L 2300/112; A61L 2300/25; A61L 2300/414; A61L 2430/02

(57) ABSTRACT

A bone substitute material for bone and dental surgery, includes: i) a solid support made from at least one phosphocalcic compound having free hydroxyl groups on the surface, and ii) a quantity of a mimetic peptide of human BMP-2 protein, having a sequence KX₁PKX₂ Z₁Z₂X₃PTEX₄SAISMLYL (SEQ ID No. 3) in which X₁, X₂, X₃ and X₄ are nonpolar amino acids, identical or different, and Z₁ and Z₂, identical or different, represent a cysteine or serine residue, the quantity of mimetic peptide of BMP-2 protein being covalently grafted by the N-terminal end of same to the hydroxyl groups, with a density lower than 100×10⁻¹² mol/mm² surface area of the solid support. The material, of which the osteoinductive properties are expressed quickly and strongly, is applicable to all fields of bone surgery. A method for producing the material with a controlled grafting density is also claimed.

The present invention belongs to the field of substitute materials intended for reconstructive bone surgery for hard tissues, such as bones, joints and teeth.

It relates to a bone substitute material that can be used for all bone surgery applications and that has rapidly and strongly expressing osteoinductive properties. This material is composed of a calcium phosphate support covalently functionalized, with a controlled density, with a peptide that is a mimetic of the human BMP-2 protein. A process for obtaining this material is also a subject of the invention.

Conventional treatment methods for bone tissue repair are based on surgery and comprise the insertion of implants, and also the use of filling cements. These bone substitutes, which may be resorbable or nonresorbable, preformed solid implants, or more or less pasty materials prepared from granules or from powders to be mixed with a solvent and which solidify after they have been injected into the area to be treated, are commonly produced from calcium phosphate compounds.

Indeed, these compounds are widely used in bone surgery since they provide implants with a greater mechanical flexibility compared with metal implants, and it is possible to formulate them as solid items or as filling cements. They are also appreciated for their cytological compatibility and their beneficial role in the formation of a new mineral bone matrix.

However, bone consolidation can take several months. As a result, in many cases, injuries, tissue regeneration defects, or else insufficient bone growth to obtain the desired result are observed. In order to solve this problem, research has been carried out on the acceleration of bone regeneration using bone morphogenetic proteins (BMPs).

BMPs proteins are a group of growth factors known to induce the formation of cartilage tissue (chondrogenesis) and bone tissue (osteogenesis) by differentiation of mesenchymal cells in vivo. They interact with specific receptors present at the surface of cells, which leads to their activation.

Among the various BMPs identified to date, BMP-2 has shown the best osteogenic capacities. However, while the BMP-2 protein has had a promising effect on animals, the results of clinical trials carried out on humans are controversial with regard to the effects obtained (Carragee E J, Hurwitz E L, Weiner B K. A critical review of recombinant human bone morphogenetic protein-2 trials in spinal surgery: emerging safety concerns and lessons learned. Spine J. 2011, 11, 471—Kang J D. Another complication associated with rhBMP-2. Spine J. 2011; 11, 517).

Its use also raises other problems. In particular, because the BMP-2 protein has a short half-life and is rapidly metabolized, the injection of a relatively high dose is required in order to induce sufficient osteogenesis during a lengthy treatment. This increases the risk of immune reactions during the administration to the patient, which can cause an undesirable level of toxicity. During this, its large-scale production is lengthy and very expensive. Finally and especially, the clinical trials have shown contradictory results with regard to the efficacy of a direct administration as a substance inducing the osteosynthesis process.

Two approaches are currently adopted for overcoming these difficulties. The first consists in transfecting the bmp-2 gene into mesenchymal stem cells and then in causing the BMP-2 protein to be expressed by the transgenic cells. However, the vectors containing the bmp-2 gene are mostly adenoviruses which could be harmful to the host cells. In addition, a mutation risk exists for the host's genes. The use of nonviral vectors eliminates this risk, but then the expression of the BMP-2 protein is reduced.

The second approach consists in using, in vivo, synthetic peptides with a length shorter than that of the complete protein, imitating the osteogenic activity of the BMP-2 protein. The peptides are designed on the basis of the known sequence of the protein, by looking for segments responsible for the osteogenic activity, and by optionally modifying the amino acids sequence so as to modulate, strengthen and reinforce this activity. These peptides can be brought to the areas to be treated in various ways and in various forms.

Document U.S. Pat. No. 7,754,683 describes three small peptides (less than 30 amino acids), obtained by adding chemical groups to the native sequence of BMP-2 corresponding to receptor II (20 amino acids). Their improved osteoinductive activity comes, according to the authors, from the presence of active anionic groups which promote mineralization. They can be formulated in solution and injected directly into the area to be treated, or encapsulated in gradually-releasing polymeric microspheres, or else integrated into the chain of a biodegradable polymer so as to form a biodegradable copolymer, which is introduced into the area to be consolidated. In this administration mode, the peptides, once released, are rapidly dispersed and degraded, which implies the use of amounts which are still large. Uncontrolled ectopic bone formation is to be feared and is unfortunately confirmed in such systems.

It has also been proposed to use synthetic grafts or filling cements mimicking the osteoinductive properties which occur in natural tissues. The biomaterials used in bone surgery can then contain growth factors, such as bone morphogenetic proteins (BMPs), in order to promote bone regeneration.

Document US 2007/0160681, for example, describes materials intended for bone graft, and structures for tissue engineering, which comprise a low-molecular-weight peptide (from 16 to 25 amino acids), immobilized at their surface in a proportion of 0.1 mg/cm² to 10 mg/cm². This peptide may be a cell adhesion-promoting peptide which has an RGD motif, or else a peptide which has growth factor activity, for example a segment of a protein such as BMP-2, having a sequence identical to the sequence of the native protein. The authors observe notable effects eight weeks after implantation.

The results obtained up until now show that many parameters are involved and must be taken into account in order to obtain a stable system well recognized by the organism and which effectively and durably stimulates its osteoinductive activity. Knowledge of the active sequences of the proteins involved is not sufficient.

Indeed, the regulation of bone differentiation involves multiple parameters, in particular due to the influence of the mechanical and biochemical properties of the microenvironment. More particularly, it is known that the physiology of the cell is affected by a series of extracellular components which strongly interact with growth factors. As it happens, the cooperation between the bone substitutes and growth factors in this microenvironment has not up until now been studied. Attempts made to create bioactive materials by grafting proteins or mimetic peptide segments thereto have proved to be disappointing.

Among the reasons that can be put forward in this respect, it may be retained that macromolecular proteins which adhere to the surface of a material have a tendency to take up various tertiary structures which are not foreseeable on the basis of their initial conformation in the free state, which can lead to a reduced and nonhomogeneous biological activity owing to insufficient exposure of the active sites. This is also valid for peptides in the soluble state. During the immobilization of peptides by covalent chemical grafting, there is a high risk that incorrect folding will occur, making the active sites inaccessible.

There is therefore a need, which to date has not been met, to have a material usable in osteo surgery which performs the role of a bone substitute and which makes it possible at the same time to promote osteoinduction and to accelerate bone formation, independently of the particular physical condition of each patient, without any immune or toxic risk.

The authors of the present invention have considered that, for a particular sequence of the peptide, the nature of the support will play a facilitating, or conversely repressive, role in the osteoinductive activity. The condition of the surfaces of the support, the nature and the amount of the reactive sites at the surface, the density of the grafting, and the nature of the bond between the peptide and the support are definitely parameters to be taken into account in designing the bone substitute corresponding to the abovementioned requirements. The methods for preparing bone substitutes combined with growth factors strongly influence these phenomena.

It is in this context that the inventors have developed an experimental model for characterizing the microenvironment responsible for the cell differentiation of the osteoblast lineage. In the context of these studies, it has been found that a determined peptide segment can induce a high osteoinductive activity when it is covalently grafted onto a calcium phosphate material, with a particular density. Surprisingly, this peptide segment of small size (about twenty amino acids only) has a high efficacy despite an environment that is very different than its natural environment. It is a peptide mime which interacts with its receptor via bonds of ionic, hydrogen and hydrophobic types. Even more surprisingly, it has been demonstrated that the maximum activity does not occur when the peptide density increases, but for densities of peptides grafted at the surface of the material corresponding to values of about a few picomoles or a few tens of picomoles per mm².

One objective of the present invention is thus to provide a material which is implantable as a bone substitute, having an increased, stronger and more rapid osteoinductive action. Another objective of the invention is to obtain, with this material, an effect that is uniformly distributed throughout the whole area of healing, and which lasts for a sufficiently long period of time to ensure complete consolidation. Yet another objective of the invention is to have a bone substitute which can be easily used, both for producing various prosthetic devices and implants, and for preparing filling cements. Another objective of the invention is to have a bone substitute material grafted with a BMP-2 protein mimetic peptide, with a minimum and optimal concentration. Of course, this material must adhere to all the standards of health safety and (non)toxicity. It must also be able to be produced under economically acceptable conditions.

The above objectives are achieved by the present invention, a subject of which is a bone substitute material for bone and dental surgery, which comprises:

i) a solid support made from at least one calcium phosphate compound having free hydroxyl groups at the surface, and
ii) an amount of a mimetic peptide of the human BMP-2 protein having the sequence

| | |
|---|---|
| KIPKACCVPTELSAISMLYL, | (SEQ ID No. 1) |

or of a derivative of this peptide which is a ligand that interacts with the type II BMP cell receptor, said derivative having a sequence

| | |
|---|---|
| KX₁PKX₂ Z₁Z₂X₃PTEX₄SAISMLYL | (SEQ ID No. 3) |

in which X₁, X₂, X₃ and X₄ are identical or different nonpolar amino acids and Z₁ and Z₂, which may be identical or different, denote a cysteine or serine residue, said amount of mimetic peptide of the BMP-2 protein being covalently grafted, via its N-terminal end, to said hydroxyl groups, with a density of less than 100×10⁻¹² mol/mm² of surface area of the solid support.

The solid supports which can be used for the present invention are calcium phosphate compounds. These compounds naturally exhibit hydroxyl functions at the surface, in high but variable number depending on their exact nature. Those which are used in bone surgery are essentially hydroxyapatites and tricalcium phosphate which all have available OH functions, but other compounds could also be suitable.

The peptide is a mimetic peptide of the native human BMP-2 protein. The human BMP-2 protein is well known for its role in bone differentiation, as a ligand of the type II cell receptor. Its complete sequence was established several years ago (Zouani O F, Chollet C, Guillotin B, Durrieu M C. Differentiation of pre-osteoblast cells on poly(ethylene terephthalate) grafted with RGD and/or BMP mimetic peptides. Biomaterials. 2010 Nov. 31(32):8245-53). The peptide sequence SEQ ID No. 1 is a segment of the native protein, which comprises 20 amino acids. The peptide according to the invention may be a 20-amino acid segment that is a mimetic of the BMP-2 protein, specially selected for its osteoinductive activity. It is described as mimetic since it constitutes a ligand that interacts with the type II BMP cell receptor. It may be identical or be a homolog derived from the native peptide, namely having the same size as said native peptide, but in which certain amino acids have been replaced.

Thus, the mimetic peptide according to the invention may be the peptide defined by the sequence SEQ ID No. 1 or any derived peptide defined by the sequence SEQ ID No. 3, integrating a possible substitution of at most four amino acids, denoted X₁, X₂, X₃ and X₄, which are nonpolar amino acids that are identical to or different from one another. The nonpolar amino acids are in particular the natural amino acids chosen from glycine, alanine, valine, leucine, methionine or isoleucine. It is, for example, possible to choose a combination where X₁=Ile, X₂=Ala, X₃=Val and X₄=Leu; or else X₁=Leu, X₂=Val, X₃=Leu and X₄=Ala.

The derivatives according to the present invention are such that they retain the property of binding the type II BMP cell receptor possessed by the peptide of sequence SEQ ID No. 1. The preferred process for producing the mimetic peptide is chemical synthesis. Any technique known to those skilled in the art can be used. For example, the peptide can be solid-phase synthesized by the method of Krieger D E et al., Proc Nat Acad Sci U.S.A. 1976; 73(9):3160-4.

The peptide segment is covalently grafted, which means that it is immobilized on the mineral matrix of the solid support. This peptide comprises in particular the six-amino-acid peptide sequence ISMLYL, which appeared to the inventors to be decisive for the osteoinductive activity to manifest itself. As a result, it is advantageously attached, via its N-terminal end, to said hydroxyl groups of the support, generally by means of a coupling agent which will be described later.

Surprisingly, it has been found that, in order to obtain a satisfactory effect, the density of peptides grafted to the support must be less than 100×10⁻¹² mol/mm² of surface area (that is to say, for the peptide according to the abovementioned first combination, the molar mass of which is 2237 g/mol, a density less than 22.35×10⁻³ mg/cm² of surface area). The system has proved to have a marked osteoinductive activity very rapidly (within the first hours) after implantation. The grafted peptides are stable and capable of durably inducing an osteogenic induction function. It may be thought that this very rapid and strong action is linked to an optimal peptide density, but also to the preservation of a correct three-dimensional conformation. A higher density might affect the folding of the peptide and therefore its activity. It is specified that the density of grafted peptide is not zero. However, it will be possible to graft very small amounts that can be as small as 0.2 pmol/mm² of surface area, while at the same time retaining a BMP receptor-inducing activity.

However, while a very low density is able to activate the BMP receptors of the cell membrane, it is possible that it will not be sufficient to induce accelerated and strong differentiation. Consequently, according to an advantageous characteristic of the invention, the predetermined density of said peptide grafted at the surface of the solid support is less than 10×10⁻¹² mol/mm².

Preferably, it is included in the range of from 4.5×10⁻¹² mol/mm² to 7.5×10⁻¹² mol/mm². More preferably, this molar density may be about 5.9×10⁻¹² mol/mm², which corresponds, for the peptide of sequence SEQ ID No. 2, to a mass density of about 1.32×10⁻³ mg/cm².

It is, moreover, noted that, since the exact nature or the structure of the support material can play a significant role on the peptide activity, it may be that the optimal grafting densities for different materials are different: by virtue of the present invention, those skilled in the art are able to control the grafting of the peptide at a biologically optimal predetermined density. This possibility follows from the two-step grafting mode which will be described in detail later, and which is also a subject of the present invention.

According to an advantageous characteristic of the material according to the invention, a cysteine is attached at the N-terminal end of the peptide. In such a way, a thiol function is available at the end of the mimetic peptide, which will be able to easily react with a site present on the solid support, inducing, by the same token, the correct orientation of the peptide during the grafting. In order for this terminal cysteine to have exclusivity of the grafting reaction, it is preferable for other amino acids present in the peptide not to react with the attachment site of the solid support.

Consequently, according to another advantageous characteristic of the material according to the invention, in said mimetic peptide, Z₁ and Z₂ are two serine residues. This substitution has proved to be all the more advisable since it has made it possible to avoid the spontaneous formation of peptide layers at the surface of the support. This phenomenon, attributed to the formation of disulfide bridges between the cysteines of various peptides, has been identified as a significant limitation to the accessibility of the peptide, and has thus been overcome by the present invention.

Thus, in one preferred variant, the peptide containing the mimetic peptide according to the invention, increased by one linking cysteine (forming a linker), has a sequence of 21 amino acids, in accordance with SEQ ID No. 2:

Nterminus-CKX₁PKX₂SSX₃PTEX₄SAISMLYL-Cterminus.

This peptide is linear and comprises the six-amino-acid sequence ISMLYL.

According to an advantageous embodiment of the invention, the peptide is bonded to the solid support by means of a bifunctional coupling agent comprising a surface-functionalizing agent combined with a linking agent. Said bifunctional coupling agent may comprise an organosilane, preferably (3-aminopropyl)triethoxysilane (denoted APTES), which can be combined with N-succinimidyl-3-maleimidopropionate (also called crosslink SMP). In a known manner, a spacer may be introduced. Finally, according to this embodiment, the mimetic peptide is bonded to the solid support by means of a cysteine attached at its N-terminal end and of a bifunctional coupling agent comprising a surface-functionalizing agent combined with a linking agent.

The solid supports used to produce the material which is the subject of the present invention are based on calcium phosphate compounds, which are capable of being used in the field of bone surgery, including in dentistry. Their chemical structure is such that they have free hydroxyl groups at the surface, although in variable proportions. The solid supports can consist of a single compound, for example a hydroxyapatite, or can have a two-phase composition, as is the case with mixtures of hydroxyapatite and calcium phosphate.

Consequently, according to one particular embodiment of the material which is the subject of the present invention, said at least one calcium phosphate compound is hydroxyapatite (HAP), tricalcium phosphate (TCP), or a mixture thereof. Other calcium phosphate materials known to those skilled in the art may also be used. It is noted that additives or other ingredients may be added to the calcium phosphate compound, either for the needs of the manufacturing process, or for therapeutic purposes (vitamins, antibiotics for example). The addition of these additives, which are always used in minor amounts, is known and will not be the subject of a particular description in the context of the present invention.

Particularly advantageously, it has been found that the material grafted with the mimetic peptide which is the subject of the invention can be obtained from a solid support, regardless of its degree of fractionation and its crystalline form. It can in particular be obtained from a porous crystalline support, such as a ceramic, but also from an amorphous support such as a powder of a calcium phosphate compound, or from calcium phosphate nanoparticles.

According to an embodiment of the invention, the solid support is a ceramic in the form of granules or of blocks. In this case, the size of the objects subjected to the grafting may be of granule type (i.e. of about one to a few millimeters) or of the size of a block (i.e. of about a few millimeters or about one centimeter). This characteristic is particularly advantageous for preparing materials according to the invention in the form of preformed solid blocks, which can replace a graft to be implanted in order to fill a sizeable gap in the bone structure. It also opens up the possibility of an application in tissue engineering, where the material will be able to be used as a support material for tissue growth before implantation (called a scaffold), in order to promote bone regeneration.

It should be emphasized that, unexpectedly and very advantageously, the material obtained in the form of a solid block has an osteoinductive activity that is just as high as that of the more fractionated forms (powder or granules), indicating that the peptide has been grafted in the body of the block. It could therefore be that, for a ceramic compound having a porosity of between 55% and 75% by volume with pores of 300 to 500 microns, the surface of the porosity remains deeply accessible to the peptide, despite the sensitivity of this type of molecule to the various strong physical and chemical interactions that were expected in such an environment.

According to another embodiment of the invention, the solid support is in the form of an amorphous powder or of nanoparticles. In this case, the size of the particles constituting the solid support may be a few micrometers for the powders, and may range from a few tens of nanometers up to approximately 200 nm for the nanoparticles. The surface area exhibiting hydroxyl groups available to react with the peptide of interest is therefore very large.

Advantageously, the material according to the invention may be provided with a solid support in the form of an amorphous powder or of nanoparticles in aqueous suspension. Bone-filling cements and nanogels (gels of nanoparticles in suspension) can thus be formulated. These materials may be stored in this form and used directly by the practitioner.

Finally, for the preparation of the innovative material, it will be possible to use a solid support which is already known and used by surgeons, who will thus not have to modify their operating protocols. In that way, surgeons have at their disposal a range of materials which allow them to choose the most appropriate form for the surgical procedure envisioned, no particular adaptation being necessary compared with the products that they are used to using. This represents a not insignificant advantage of the invention.

Whatever the solid support chosen, it offers at the surface a large amount of OH sites that can be grafted.

The material described above, comprising a bone substitute having free hydroxyl functions, combined with its BMP-2 mimetic peptide, covalently immobilized via its N-terminal end, induces rapid differentiation and osteoblast proliferation. Moreover, it induces a strong interaction between bone marrow cells such as mesenchymal stem cells, osteoblasts and vascular cells. Cell clusters are formed which attest to a strong regenerating power of these grafted materials.

The results obtained therefore attest to the creation of a cell microenvironment comprising the BMP-2 mimetic peptides, which promotes bone regeneration in a short period of time. It can be used in all the fields of bone surgery (musculoskeletal and dental), in particular in orthopedics, in neurosurgery, in maxillofacial surgery, in stomatology, in oncology, implantology, vertebroplasty, or the like. The material may also be used as it is, or else combined with other types of supports and biomaterials, for example with composite materials with a polymer matrix.

The present invention is also directed toward a protocol for preparing a material constituting such a cell microenvironment. The material according to the invention can be prepared from various calcium phosphate, ceramic, amorphous powder or nanocrystal substrates, by grafting a peptide as previously defined, with the proviso that the process used makes it possible to control the amount of BMP-2 protein mimetic peptide which is covalently grafted by its N-terminal end to said hydroxyl groups, such that this amount is less than 100 pmol/mm² of surface area of the calcium phosphate compound. To do this, a process has been developed which is also a subject of the present invention.

Thus, according to the invention, what is claimed is a process for preparing a bone substitute material as claimed in one of the preceding claims, comprising i) a solid support made from at least one calcium phosphate compound having free hydroxyl groups at the surface, and ii) an amount of a mimetic peptide of the human BMP-2 protein having the sequence

| | |
|---|---|
| KIPKACCVPTELSAISMLYL, | (SEQ ID No. 1) |

or of a derivative of this peptide which is a ligand of the type II cell receptor, said derivative having a sequence

| | |
|---|---|
| KX₁PKX₂ Z₁Z₂X₃PTEX₄SAISMLYL | (SEQ ID No. 3) |

in which X₁, X₂, X₃ and X₄ are identical or different nonpolar amino acids and Z₁ and Z₂, which may be identical or different, denote a cysteine or serine residue, the process essentially comprising the steps consisting in:
a) reacting a part of the hydroxyl groups of said calcium phosphate compound with a functionalizing agent and then with a linking agent so as to obtain a solid support having a predetermined density of modified sites at the surface,
b) reacting the solid support thus modified with a solution containing said peptide in excess so as to obtain a material having said predetermined density of less than 100×10⁻¹² mol/mm² (i.e. 100 pmol/mm²) of sites grafted with said peptide.

Step a), which comprises two successive reactions, makes it possible to carry out a modification of a given number of free hydroxyl sites present at the surface of the calcium phosphate compound, using a bifunctional coupling agent made up of a functionalizing agent with which is combined a linking agent that will then be capable of reacting with the peptide of interest. Step b) carries out the reaction between the linking agent and the mimetic peptide. The density of hydroxyl groups modified in step a) is controlled by controlling the reaction conditions, in particular the successive incubation times of the support with the two reactive agents. Since the various calcium phosphate compounds naturally have greater or fewer free OH sites, the reaction time will be adjusted so that a predefined desired amount of sites is modified. Secondly, the peptide of interest is grafted, under reaction conditions such that the previously modified sites will react with the peptide. Even if not all the modified sites of the support react with the mimetic peptide, it nevertheless remains the case that a certain defined proportion of modified sites is grafted with the peptide, this proportion being reproducible under fixed operating conditions. It is therefore in step a) that the density of grafting that will finally be obtained is determined. The density of modified sites at the end of step a) is generally greater than the density of sites grafted with the peptide at the end of step b).

The conditions of the process are therefore adjusted so as to obtain a solid support having a predetermined density of modified and then grafted sites at the surface. Under these conditions, it appears that the formation of a monolayer of peptides is promoted, with controlled steric hindrance and without marked interference between neighboring peptide groups, such that the folding of the peptides is not affected.

According to a preferred characteristic of the process which is the subject of the invention, in step a), some of the hydroxyl groups of said calcium phosphate compound are reacted with a functionalizing agent and then with a linking agent so as to obtain, in step b), a solid support having a predetermined density of sites grafted at the surface of less than 10×10⁻¹² mol/mm². Preferably, the predetermined density of sites grafted at the surface ranges from 4.5×10⁻¹² mol/mm² to 7.5×10⁻¹² mol/mm².

A support modified with an assembly constituting a bifunctional coupling agent is thus created. It is formed, for example, from a compound chosen from organosilanes, such as (3-aminopropyl)triethoxysilane (or APTES), which can be combined with a linking agent of the succinimidyl family, such as N-succinimidyl 3-maleimidopropionate (crosslink SMP) or a dendron containing several maleimide functions.

In a particular implementation of the process which is the subject of the invention, in step a), the solid support is placed in a solution of (3-aminopropyl)triethoxysilane, and then in a solution of N-succinimidyl-3-maleimidopropionate, so as to obtain said predetermined density of modified hydroxyl sites at the surface of said solid support. For example, a solution of SMP at 2×10⁻³M in DMF can be used.

According to another possible characteristic of the process which is the subject of the invention, in step b), the modified solid support is placed in an aqueous solution containing said peptide at a molarity of between 10⁻⁵M and 10⁻²M, for 15 to 30 hours. Advantageously, it is possible to work with an aqueous solution of peptide at 10⁻³M, which is then in great excess. The incubation time is sufficient for the reaction of the peptides with the modified sites in step a) to be at a maximum.

Preferably according to the invention, said peptide is covalently grafted onto the sites modified in step a) by its N-terminal end. If the peptide has a cysteine in the N-terminal position, it is the thiol group of the cysteine which reacts with the maleimide part of the crosslink SMP.

According to a preferred embodiment of the invention, before each of the two reactions of step a), the solid support is dried at a temperature of about 70° C. to 100° C. The drying is carried out in a thermostated oven and takes a few hours. Any trace of solvent and in particular of water potentially present around and, where appropriate, in the porosity of the solid support, is thus removed, thereby improving the yield of the reactions with a high reproducibility.

Indeed, in step a), the reaction of the functionalizing agent with the support material in the presence of traces of water leads to the formation of a polysiloxane network which disrupts the subsequent attachment of the peptides. The occurrence of this three-dimensional network of polymeric chains can be prevented by heating the material. In certain variants of the process, it is possible to operate under a secondary vacuum defined as corresponding to pressures from 10⁻³ to 10⁻⁷ mbar (i.e. from 0.1 to 10⁻⁵ pascal). These operating modes thus play a notable role in obtaining a monolayer.

According to an advantageous characteristic of the innovative process, after step b), rinsing with water of the bone substitute material obtained is carried out until there is complete elimination of the excess peptides, which includes here the peptides that have not been grafted but have just been adsorbed. In that way, the peptides which have not formed a covalent bond with the linking agent are eliminated. This rinsing phase may be of appreciable importance in the osteoinductive activity that the material will exhibit. In this respect, the hypothesis has been put forward that the grafted sites might be masked by compounds that have been adsorbed but not covalently immobilized, in particular excess peptides, that the patient's body would have to eliminate before the grafted peptide segments become accessible and can start to act.

The grafting process can also be carried out in an aqueous medium, according to the same approach in two major steps. In this case, the process is carried out as follows: in step a): addition of the functionalizing agent (APTES) in an acidic medium, then addition of a linking agent (SMP), rinsing and drying; then in step b): addition of the BMP-2 peptide solutions, and rinsing for 1 week.

According to a preferred characteristic of the process for preparing the bone substitute material according to the invention, the calcium phosphate compound is hydroxyapatite (HAP), tricalcium phosphate (TCP), or a mixture thereof. Those skilled in the art know how to produce such compounds and mixtures thereof. It is also known practice to choose one or the other depending on the intended use. Thus, said solid support can be chosen from:
a ceramic formulated in granules or in blocks, so as to obtain said bone substitute material in the same form,
an amorphous powder capable of being suspended in an aqueous phase so as to obtain said bone substitute material in the form of a filling cement,
nanoparticles capable of being suspended in an aqueous phase so as to obtain said bone substitute material in the form of a filling gel.

The present invention will be understood more clearly, and details relating thereto will emerge, by virtue of the description that will be given of certain embodiments, in relation to the appended figures, in which:

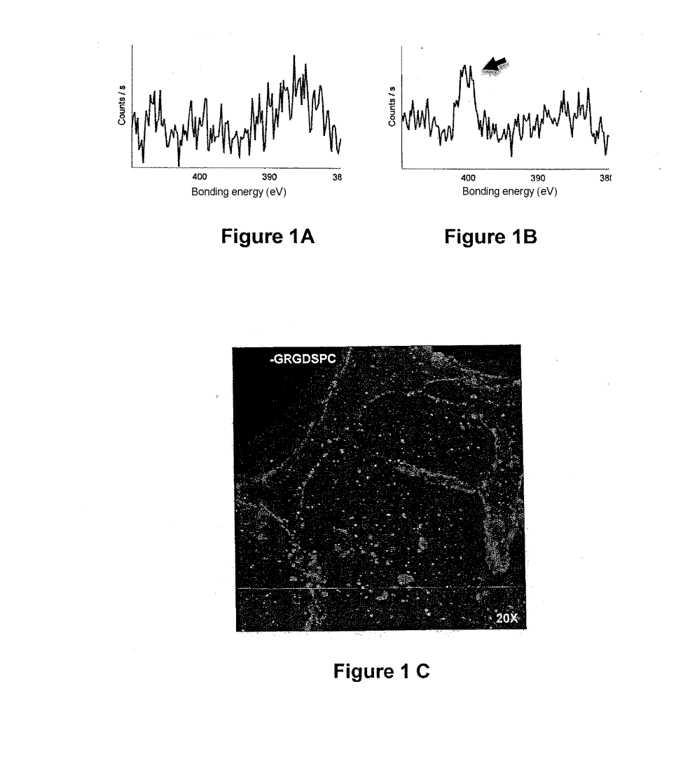

FIG. 1 shows the nitrogen atomic spectrum of a material grafted with a peptide, which is the subject of the invention (FIG. 1B), and of the solid support before grafting (FIG. 1A).

FIG. 1C is a fluorescence microscopy image illustrating the presence of the peptide grafted onto this same material.

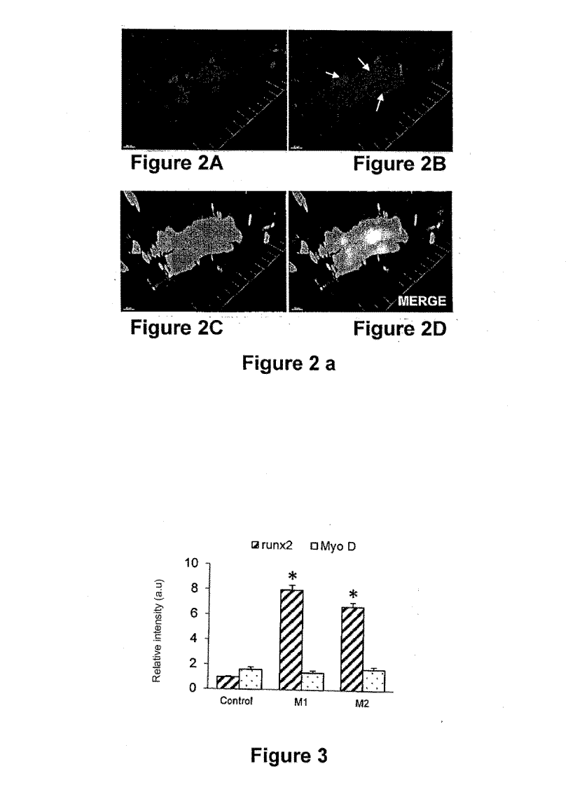

FIG. 2 shows confocal microscopy images illustrating the differentiation of human bone marrow mesenchymal stem cells after 48 h in contact with a material according to the invention.

FIG. 3 is a graph illustrating the level of expression of the Runx2 factor and of the MyoD factor after 62 h of contact of the same cells with two materials according to the invention.

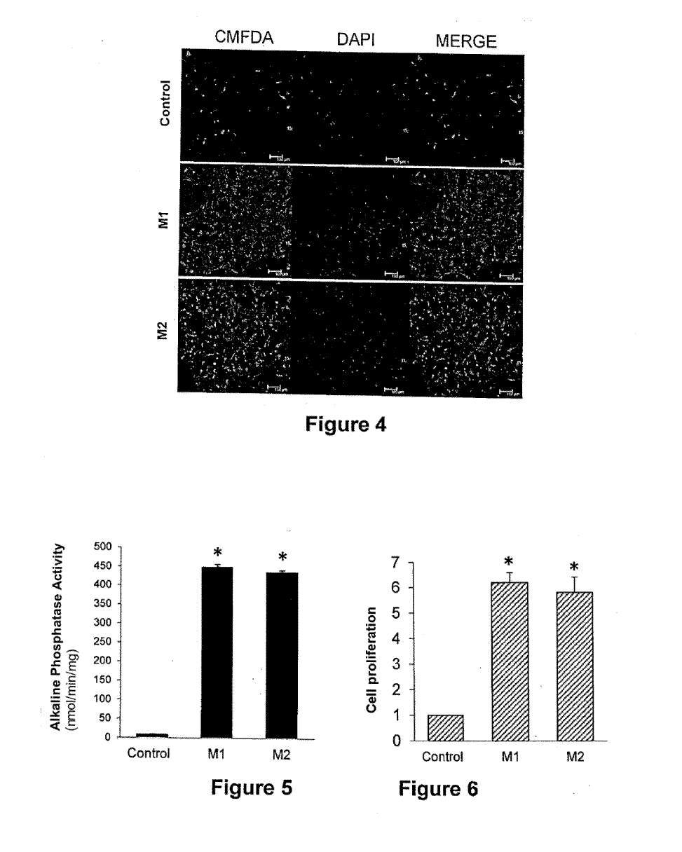

FIG. 4 shows confocal microscopy images illustrating the differentiation of pre-osteoblast cells after 48 h in contact with two materials according to the invention.

FIG. 5 is a quantitative representation of alkaline phosphatase activity after 48 hours of culture of the same cells with the same two materials.

FIG. 6 is a graph illustrating the level of expression of the Runx2 factor after 48 h of contact of the same cells with the same two materials according to the invention.

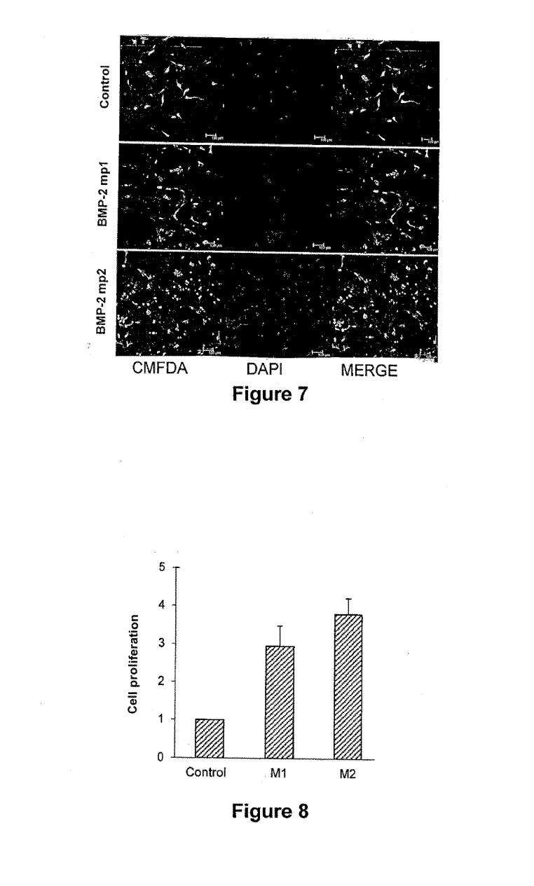

FIG. 7 represents confocal microscopy images of endothelial cells cultured for 48 hours in contact with two materials according to the invention.

FIG. 8 is a graph showing the proliferation of the endothelial cells after 48 hours of cell culture in contact with the same two materials.

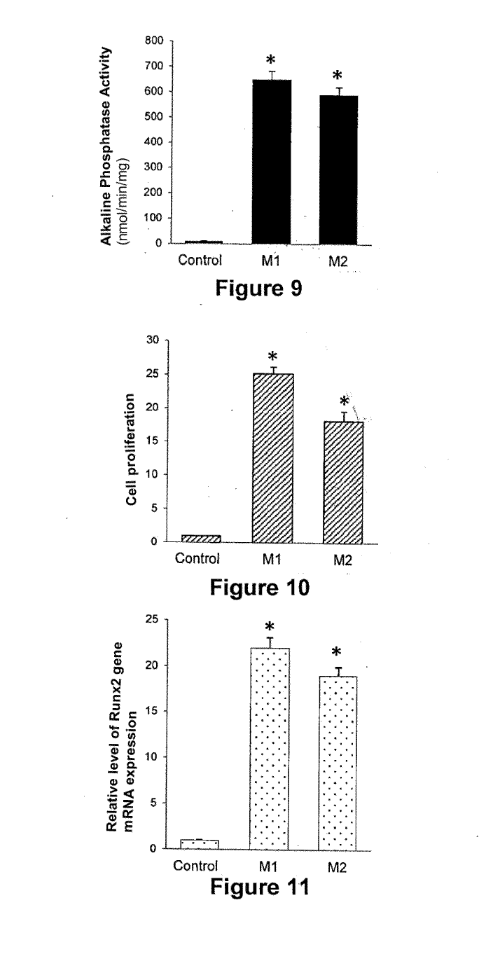

FIG. 9 is a graph showing the alkaline phosphatase activity of cell tricultures after 62 hours in contact with two materials according to the invention.

FIG. 10 is a graph showing the proliferation of osteoblast precursors after 62 hours of cell culture on these same materials.

FIG. 11 is a graph illustrating the level of expression of the Runx2 factor after 62 h of contact of the same cells with the same two materials.

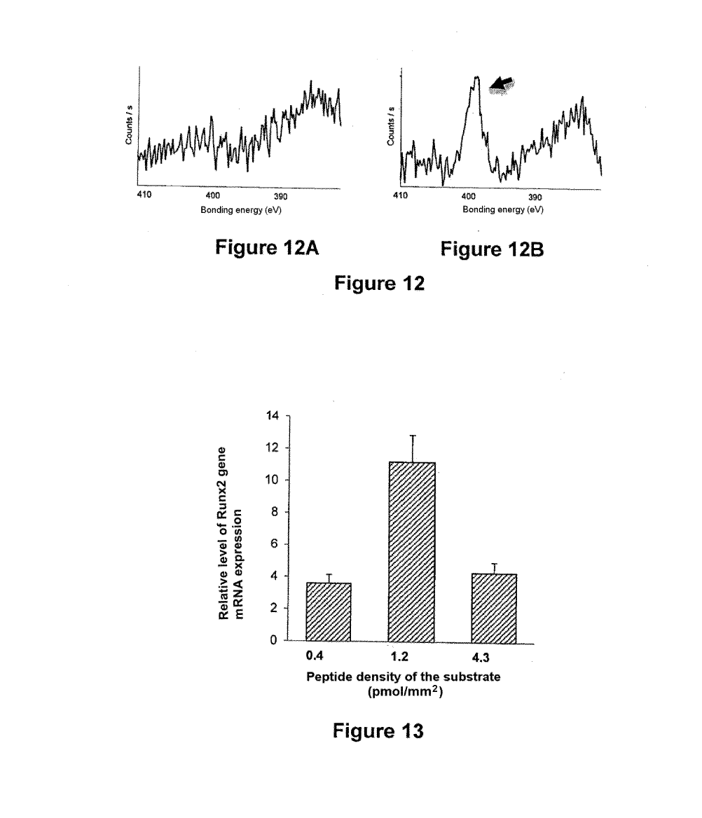

FIG. 12 shows the nitrogen atomic spectrum of a nanoparticulate material grafted with a peptide which is the subject of the invention (FIG. 12B) and of the solid support before grafting (FIG. 12A).

FIG. 13 represents the level of expression of the Runx2 factor by RT-PCR as a function of the density of the peptide P1 grafted at the surface of a PET model support.

### EXAMPLE 1

### Ceramic Material Grafted with a Mimetic Peptide P1

### Support:

In the embodiment presented hereinafter, the solid support is a ceramic material containing 65% of hydroxyapatite and 35% of TCP. The material has a porosity of about 65% to 75%. It is commercially available, for example under the brand name Ceraform® from Teknimed. It is used in the form of 6 mm-sided cubes.

### Peptide:

The peptide P1 having the sequence: CKIPKASSVPTELSAISMLYL.

It is a linear peptide which ensues from SEQ ID No. 3:

KX₁PKX₂Z₁Z₂X₃PTEX₄SAISMLYL, increased by one cysteine, with X₁=Ile, X₂=Ala, X₃=Val and X₄=Leu, and Z₁═Z₂=Ser.

### Procedure (Step a)

(1) Place the support under vacuum (10⁻⁵ torr) at 100° C. for 16 hours.
(2) Under an argon atmosphere, place the support with APTES at 10⁻²M in anhydrous hexane for 2 hours.
(3) Rinse with sonication.
(4) Dry under vacuum (10⁻⁵ torr) at 70° C. for 16 h.
(5) Add the linking agent SMP at a concentration of 2×10⁻³M in DMF and incubate for 2 hours under an argon atmosphere.
(6) Rinse several times in DMF, then dry at 70° C. under vacuum (10⁻⁵ torr).
A modified support is obtained.

### Procedure (Step b)

(7) Add the peptide solution of the peptide P1 (10⁻³M) to the modified support thus obtained and incubate under a normal atmosphere at ambient temperature for 18 h.
(8) Carry out rinsing for one week.

The material M1 grafted with P1 is obtained (6 mm-sided cube).

### Characterization: Presence of the Peptide Sequence

The atomic composition of the grafted ceramic M1 was compared with that of the nontreated ceramic (solid support taken as reference). The measurements were carried out by X-ray photoelectron spectroscopy (table 1).

TABLE 1

| Atom | Reference (atomic %) | Material M1 (atomic %) |
|---|---|---|
| P2p | 10.5 | 10.55 |
| C1s | 33.5 | 34.52 |
| Ca2p | 15.59 | 13.93 |
| O1s | 39.4 | 38.39 |
| N1s | not detected | 2.61 |

The nitrogen spectrum (N1s) at the surface of the two materials (M1 and reference), obtained by X-ray photoelectron spectroscopy, is shown in FIGS. 1A and 1B.

The results show the appearance, in the material M1, of a nitrogen spectrum at approximately 3%, proving the presence of peptide sequence on the ceramic support.

### In the Body of the Support:

Cubes of the material M1 obtained as described above were fractionated into a granulated material less than 1 mm in diameter, and subjected to rinsing for 1 month. Various points recorded by X-ray photoelectron spectroscopy showed the presence of nitrogen in an amount of approximately 1.5%, whereas no trace of nitrogen is detected in the nonmodified ceramic supports.

There is therefore deep grafting of the ceramic support M1.

### Characterization: Density of Grafted Peptide

The ceramic material M1 was characterized in order to determine the exact density of peptides grafted at the surface of the solid support. For this, a fluorescent peptide P1f was prepared and grafted to the solid support so as to obtain a material M1f. The peptide P1f is the peptide P1 augmented at its C-terminal end with a fluorochrome, in this case fluorescein isothiocyanate (abbreviated to FITC).

The average density of peptide P1f at the surface of the material M1, measured by fluorescence microscopy, is 5.9 pmol/mm².

### EXAMPLE 2

### Ceramic Material Grafted with a Mimetic Peptide P2

### Support:

The solid support is a ceramic cube (65% of hydroxyapatite and 35% of TCP), identical to that of example 1.

### Peptide:

The peptide P2 having the sequence: CKLPKVSSLPTEASAISMLYL.

It is a peptide in accordance with the peptide having the sequence:

KX₁PKX₂Z₁Z₂X₃PTEX₄SAISMLYL (SEQ ID No. 3), increased by one cysteine, with X₁=Leu, X₂=Val, X₃=Leu and X₄=Ala, and Z₁═Z₂=Ser.

### Procedure:

The grafting protocol is identical to that of example 1.

The grafted material M2 is obtained.

### Characterization of M2:

The results are similar to those obtained for the material M1.

### EXAMPLE 3

### Osteogenic Activity of the Grafted Ceramic Materials M1 and M2

The activity of the materials M1 and M2 described and prepared in accordance with the examples above was evaluated in contact with three cell types separately and together within the same cell culture. The three cell types are the cells predominantly present in bone marrow. They are mesenchymal stem cells, pre-osteoblast cells and endothelial cells.

1—Activity with Respect to Mesenchymal Stem Cells

Human bone marrow mesenchymal stem cells (supplied by the company Lonza) were cultured for 96 hours on nonmodified ceramic supports and ceramic supports M1 and M2 grafted respectively with the peptides P1 and P2.

The osteogenic commitment of the cells after 48 hours of culture on the materials M1 and M2 grafted with the peptide P1 and the peptide P2 was pinpointed by confocal microscopy. The images obtained reveal the cell form by virtue of cytoplasmic labeling with CMFDA (FIG. 2C), the Runx2 master osteogenic transcription factor being labeled with an anti-Runx2 antibody, represented in FIG. 2B, and the nucleus being labeled with DAPI (FIG. 2A), and a merged image (Merge, FIG. 2D). It is noted that all the mesenchymal stem cells very strongly express the Runx2 transcription factor, attesting to commitment of the cells toward an osteoblast lineage.

A quantification of the expression level of this Runx2 factor in the materials M1 and M2 compared with a nonmodified ceramic support was established by quantification of the fluorescence from confocal images on at least 60 cells. Various regions of the material were inspected at the center and at the periphery; a homogeneous cell differentiation effect was observed. Labeling of the myogenic transcription factor MyoD, attesting to a myoblast cell commitment, was carried out during the cell labeling as a negative control. The measurements were carried out 62 hours after bringing the cells into contact with the materials M1 and M2. The results obtained are reported in FIG. 3. An increase of 80% in the expression of the Runx2 factor is found for the material M1 and an increase of 68% is found for the material M2, relative to the reference material. No trace of increase in the expression of the transcription factor MyoD was observed (*P<0.01).

2—Activity with Respect to Pre-Osteoblast Cells

Pre-osteoblast cells (the MC3T3-E1 line, sold by ATCC) were cultured on the materials M1 and M2. The nongrafted solid support is taken as reference.

After 48 hours of cell culture on the materials M1 and M2, the osteoblast precursors were demonstrated by cytoplasmic labeling with CMFDA. The images obtained by confocal microscopy are shown in FIG. 4.

The alkaline phosphatase activity was also determined. It is multiplied by approximately 400 in the pre-osteoblast cells on contact with the materials M1 and M2, compared with the activity of the cells cultured on a nonmodified ceramic support (FIG. 5), revealing a strong expression of alkaline phosphatase. A significant difference is observed between the cells cultured on the nonmodified ceramic support and the grafted materials M1 and M2 (*P<0.001).

Proliferation of the osteoblast precursors was observed. After 48 hours of cell culture, the splitting of cell populations is six times greater on the supports M1 and M2, as shown by FIG. 6 (*P<0.005).

These results demonstrate a strong bone regenerating power developed by the materials M1 and M2, grafted with the BMP-2 protein mimetic peptides, P1 and P2.

3—Activity with Respect to Endothelial Cells

Endothelial cells (cell type present in bone marrow in the vessels) were brought into contact with the modified materials M1 and M2, and with the nonmodified ceramic support taken as reference. After 48 hours of culture of the endothelial cells (HUVEC, ATCC), proliferation of the cells is observed (FIG. 7), which is 3 times greater on the supports M1 and M2 than on the nonmodified ceramic support (*P<0.005) (FIG. 8).

4—Activity with Respect to Bone Marrow Cells

Finally, the effect of the materials M1 and M2 in simultaneous contact with the three cell types above was evaluated in order to predict their behavior once implanted in vivo.

The cell tricultures comprising human bone marrow mesenchymal stem cells, osteoblast precursors and endothelial cells were brought into contact with the materials M1 and M2 and with a nongrafted support taken as reference, for 62 hours. The images obtained by confocal microscopy (not reproduced) show the formation of very large cell clusters (or cell networks).

The alkaline phosphatase activity was determined after 62 hours of culture (FIG. 9). A significant difference is observed between the cells cultured on nonmodified ceramic supports and the grafted materials M1 and M2, since it is 650 times higher (*P<0.01). The expression of the Runx2 osteogenic transcription factor was determined by measuring the intensity of its fluorescence in the cell triculture after 62 hours (*P<0.05). It is strong: twenty times greater than the reference (FIG. 11). The cell proliferation is increased, with splitting of the cell populations that is twenty times greater in contact with the materials M1 and M2 than in contact with the nongrafted support (*P<0.005). This demonstrates a strong regenerating power of the materials M1 and M2 (FIG. 10).

All these results demonstrate the rapidity and the strength of the osteoinductive activity of the ceramic supports grafted with the BMP-2 protein mimetic peptides according to the invention.

### EXAMPLE 5

### Nanoparticulate Material Grafted with the Mimetic Peptide P1

In the embodiment presented hereinafter, the material obtained is a gel of hydroxyapatite nanoparticles in suspension in water.

### Support:

The solid support consists of hydroxyapatite nanoparticles (nanorods of submicronic size) which are in the form of a powder, after lyophilization. They are dense (nonporous) particles characterized by a high specific surface area.

### Peptide:

The peptide P1 as previously described is used.

### Procedure:

The grafting protocol is identical to that of example 1. At the end of steps a) and b), the grafted nanoparticulate material NP1 is obtained in the form of a powder.

The grafted powder is then suspended in water, so as to obtain a gel of hydroxyapatite nanoparticles in suspension in water (5 mg/ml). It is composed of nanorods in water, with a solids content of about 30%.

### Characterization: Presence of the Peptide Sequence

The atomic composition of the grafted nanoparticles NP1 was compared with that of nontreated nanoparticles (solid support taken as reference). The measurements were carried out by X-ray photoelectron spectroscopy (table 2).

TABLE 2

| Atom | Reference (atomic %) | Material NP1 (atomic %) |
|---|---|---|
| P2p | 11.4 | 11.25 |
| C1s | 19.9 | 20.15 |
| Ca2p | 17.39 | 18.83 |
| O1s | 48.5 | 48.89 |
| N1s | not detected | 3.61 |

The nitrogen spectrum (N1s) at the surface of the two materials (NP1 and reference), obtained by X-ray photoelectron spectroscopy, is shown in FIGS. 12A and 12B.

The results show the appearance, in the material NP1, of a nitrogen spectrum at approximately 4%, proving the presence of peptide sequence on the nanoparticulate support.

### Characterization: Density of Grafted Peptide

The presence of grafted peptides at the surface of the material NP1 was determined by fluorescence microscopy using fluorescent peptides P1. The results demonstrate that covalent grafting onto the support NP1 is indeed obtained. In addition, the transmission electron microscopy images obtained of the ceramic nanoparticles at the various stages of the grafting protocol show that the grafting method has not affected the sizes of the nanoparticles. More generally, it is the possibility of peptide grafting onto nanoparticulate supports which is validated.

### EXAMPLE 6

### Optimization of the Density of Grafting on a Model Support

It was demonstrated, on a synthetic model support, that it is possible to control the density of grafting on the sites of the support, and that the optimal density can thus be predetermined.

The peptide P1 was covalently grafted onto a model support, at three different densities. The support is made of polyethylene terephthalate (PET). The densities are 0.4 pmol/mm², 1.2 pmol/mm² and 4.3 pmol/mm². The expression level of the Runx2 factor by RT-PCR as a function of the density of the peptide P1 grafted at the surface of a model support was measured for these three densities (FIG. 13). The best expression level of the Runx2 gene is obtained for 1.2 pmol/mm² of peptide density, which corresponds to a maximum cell differentiation activity for the model support.

The activity obtained is also stable over time (after 1 week of culture): at 96 h of culture, a continuous increase in the expression of the Runx2 factor (approximately 42%) is observed.

### EXAMPLE 7

### Optimization of the Density of Grafting on a Calcium Phosphate Support

The peptides P1 and P2 were covalently grafted onto a calcium phosphate support, at three different densities. The solid support is a ceramic cube (65% of hydroxyapatite and 35% of TCP), identical to that of example 1. Grafting densities of 1.1 pmol/mm², 3.2 pmol/mm² and 4.9 pmol/mm² were obtained. It is thus demonstrated that it is possible to modulate and control as desired the degree of grafting of the peptides of interest onto the calcium phosphate supports.

The expression level of the Runx2 factor measured by RT-PCR, the cell proliferation and the alkaline phosphatase activity as a function of the density of the peptides grafted at the surface of this support were measured for the densities 3.2 and 4.9 pmol/mm². The Runx2 gene expression, cell proliferation and alkaline phosphatase activity results showed a marked cell differentiation activity from the viewpoint of the low grafted-peptide densities.

1-15. (canceled)

16. A bone substitute material for bone and dental surgery, comprising:
i) a solid support made from at least one calcium phosphate compound having free hydroxyl groups at the surface, and
ii) an amount of a mimetic peptide of the human BMP-2 protein having the sequence

| | |
|---|---|
| KIPKACCVPTELSAISMLYL, | (SEQ ID No. 1) |

or of a derivative of this peptide which is a ligand that interacts with the type II BMP cell receptor, said derivative having a sequence

| | |
|---|---|
| KX₁PKX₂Z₁Z₂X₃PTEX₄SAISMLYL | (SEQ ID No. 3) |

in which X₁, X₂, X₃ and X₄ are identical or different nonpolar amino acids and Z₁ and Z₂, which may be identical or different, denote a cysteine or serine residue, said amount of mimetic peptide of the BMP-2 protein being covalently grafted, via its N-terminal end, to said hydroxyl groups, with a density of less than 100×10⁻¹² mol/mm² of surface area of the solid support.

17. The material as claimed in claim 16, wherein the density of said peptide grafted at the surface of the solid support is less than 10×10⁻¹² mol/mm².

18. The material as claimed in claim 17, wherein
the density of said peptide grafted at the surface of the solid support is included in the range of from 4.5×10⁻¹² mol/mm² to 7.5×10⁻¹² mol/mm².

19. The material as claimed in claim 16, wherein, in said peptide, Z₁ and Z₂ are two serine residues.

20. The material as claimed in claim 16, wherein the peptide is bonded to the solid support by means of a cysteine attached at its N-terminal end and of a bifunctional coupling agent comprising a surface-functionalizing agent combined with a linking agent.

21. The bone substitute material as claimed in claim 16, wherein said peptide is the peptide of sequence SEQ ID No. 3, in which:
X₁=Ile, X₂=Ala, X₃=Val and X₄=Leu.

22. The bone substitute material as claimed in claim 16, wherein said peptide is the peptide of sequence SEQ ID No. 3, in which:
X_{′}=Leu, X₂=Val, X₃=Leu and X₄=Ala.

23. The material as claimed in claim 16, wherein said at least one calcium phosphate compound is hydroxyapatite, tricalcium phosphate, or a mixture thereof.

24. The material as claimed in claim 16, wherein the solid support is chosen from a ceramic in the form of granules or of blocks, an amorphous powder, or calcium phosphate nanoparticles.

25. A process for preparing a bone substitute material as claimed in claim 16, comprising: i) a solid support made from at least one calcium phosphate compound having free hydroxyl groups at the surface, and ii) an amount of a mimetic peptide of the human BMP-2 protein having the sequence

| | |
|---|---|
| KIPKACCVPTELSAISMLYL, | (SEQ ID No. 1) |

or of a derivative of this peptide which is a ligand of the type II cell receptor, said derivative having a sequence

| | |
|---|---|
| KX₁PKX₂Z₁Z₂X₃PTEX₄SAISMLYL | (SEQ ID No. 3) |

in which X₁, X₂, X₃ and X₄ are identical or different nonpolar amino acids and Z₁ and Z₂, which may be identical or different, denote a cysteine or serine residue, the process comprising the steps of:
a) reacting a part of the hydroxyl groups of said calcium phosphate compound with a functionalizing agent and then with a linking agent so as to obtain a solid support having a predetermined density of modified sites at the surface; and
b) reacting the solid support thus modified with a solution containing said peptide in excess so as to obtain a material having a predetermined density of less than 100×10⁻¹² mol/mm² of sites grafted with said peptide.

26. The process for preparing a bone substitute material as claimed in claim 25, wherein, in step a), some of the hydroxyl groups of said calcium phosphate compound are reacted with a functionalizing agent and then with a linking agent so as to obtain, in step b), a solid support having a predetermined density of sites grafted at the surface of less than 10×10⁻¹² mol/mm².

27. The process for preparing a bone substitute material as claimed in claim 26, wherein, in step a), some of the hydroxyl groups of said calcium phosphate compound are reacted with a functionalizing agent and then with a linking agent so as to obtain, in step b), a solid support having a predetermined density of sites grafted at the surface ranging from 4.5×10⁻¹² mol/mm² to 7.5×10⁻¹² mol/mm².

28. The process for preparing a bone substitute material as claimed in claim 25, wherein, before each of the two reactions of step a), the solid support is dried at a temperature of about 70° C. to 100° C.

29. The process for preparing a bone substitute material as claimed in claim 25, further comprising rinsing with water the bone substitute material obtained after step b) until there is complete elimination of the excess peptides.

30. The process for preparing a bone substitute material as claimed in claim 25, wherein said solid support is chosen from:
a ceramic formulated in granules or in blocks, so as to obtain said bone substitute material in the same form,
an amorphous powder capable of being suspended in an aqueous phase so as to obtain said bone substitute material in the form of a filling cement,
nanoparticles capable of being suspended in an aqueous phase so as to obtain said bone substitute material in the form of a filling gel.
